(19) United States
(12) Patent Application Publication
(10) Pub. No.: US 2007/0264291 A1
(43) Pub. Date: Nov. 15, 2007

(54) Compositions and Methods for Preventing or Reducing Plaque and/or Gingivitis Using a Bioactive Glass Containing Dentefrice

(76) Inventors: David C. Greenspan, Gainesville, FL (US); Guy LaTorre, Gainesville, FL (US); Leonard J. Litkowski, Baltimore, MD (US); Gary D. Hack, Columbia, MD (US)

Correspondence Address: HUTCHISON LAW GROUP PLLC, PO BOX 31686, RALEIGH, NC, 27612, US

(21) Appl. No.: 10/582,695

(22) PCT Filed: Dec. 17, 2004
(86) PCT No.: PCT/US04/43097

§ 371 Date: Mar. 21, 2007

Related U.S. Application Data
(60) Provisional application No. 60/530,965, filed on Dec. 19, 2003.

(51) Int. Cl. A61Q 11/00; A61K 8/02

(52) U.S. Cl. 424/401; 424/49

(57) ABSTRACT

Methods and compositions for the prevention and/or reduction of plaque, plaque build-up and/or gingivitis are provided. Bioactive glass compositions are provided which prevent or reduce plaque, plaque build-up and/or gingivitis through the use of low levels of small bioactive glass particles in amounts from about 0.25 to about 10% by weight in non-aqueous formulations. The resulting non-aqueous compositions are effective in dentifrice products and are stable and pass ISO (International Organization for Standardization) standards. Moreover, these bioactive glass containing non-aqueous compositions have unexpectedly high levels of antimicrobial activity against oral pathogens.

### BACKGROUND OF TH INVENTION

1. Field of the Invention

The present application relates to the prevention or reduction of plaque on teeth by application of bioactive glass in a non-aqueous carrier. The present application further relates to the prevention or reduction of gingivitis by application of bioactive glass in a non-aqueous carrier.

2. Description of Related Art

It is well established that tooth decay, development of plaque, plaque build-up, gingivitis, periodontal disease and other conditions of the oral cavity are associated with pathogens such as Actinobacillus actinomycetemcomitans, Porphyromonas gingivalis, Actinomyces naeslundii, and/or Streptococcus mutans, among many others. For most individuals, proper oral care, including brushing with a standard, commercial dentifrice and appropriate toothbrush along with the use of dental floss daily will maintain proper oral health. Even with proper oral health care, a significant number of persons suffer from tooth decay, plaque build-up and gingivitis that can lead to serious oral health issues. It is estimated that over 150,000,000 cavities are filled in the United States every year at a cost of over $11 billion, and that over 20% of the adult population suffers from some form of gingivitis, from mild inflammation to severe gingival bleeding.

Gingivitis is a first form of periodontal disease typically caused by the long-term effects of plaque deposits. Plaque is the sticky, colorless, film material that develops on the exposed portions of the teeth. Unremoved plaque mineralizes into a hard deposit called calculus or tartar that becomes trapped at the base of the tooth. Plaque and calculus cause mechanical irritation and inflammation while bacteria in plaque causes the gums to become infected, swollen and tender. Other causes of gingivitis may include overly vigorous brushing or flossing the teeth or other injury or trauma to the gums. The conditions and problems stemming from plaque, plaque build-up and gingivitis may eventually lead to tooth loss, and a general degradation in a patient's overall health.

In order to address the issues surrounding oral heath, the use of antibiotic mouth rinses such as Peridex® which contains chlorhexadine, or other such products, have been employed. While such products may be somewhat effective in reducing gingivitis, there are many drawbacks to using such mouth rinses for any period of time. The use of such products may result in increased resistance of the oral microorganisms to the drug in the mouth rinse, the antibiotic used has the potential of staining the teeth after prolonged use of the antibiotic and the ingredients in toothpaste used with the mouth rinse may lessen the effectiveness of the chlorhexadine, thus necessitating adherence to strict timing of the usage of the mouth rinse. In most instances, for example, the manufacturer recommends waiting at least 30 minutes after brushing to insure that all traces of toothpaste are removed. Thus, mouth rinses do not provide a satisfactory long term approach for the prevention or reduction of plaque, plaque build-up and/or gingivitis.

Other approaches to gingivitis have been tried. U.S. Pat. No. 6,190,643, issued to Stoor et al., describes methods for reducing the viability of detrimental oral microorganisms in an individual and for prevention and/or treatment of diseases caused by such microorganisms, such as dental caries and/or gingivitis, and for whitening of an individual's teeth, wherein the methods are ostensibly due to the reduction of the viability and, thus, the decrease of the number of detrimental oral microorganisms.

The method described in Stoor et al. comprises subjecting the individual's oral cavity and/or root canals to a bioactive glass, the average particle size of which is less than 100 μm. Bioactive glasses, as used in Stoor et al.; are well known in the art, and have demonstrated an ability to regenerate bone tissue when implanted into bony defects. U.S. Pat. No. 4,851,046, issued to Low et al., describes the use of particulate bioactive and biocompatible glass of relatively large particles of 90 to 710 μm for repair of periodontal osseous defects. U.S. Pat. No. 5,834,008, issued to Greenspan et al., discloses a composition for the healing of wounds and burns comprising particulates of bioactive glass and at least one topical antibiotic wherein the particle size range of the bioactive glass is less than 90 microns.

Bioactive glasses have additionally been used for other indications in the oral cavity. U.S. Pat. No. 6,086,374, issued to Litkowski et al. discloses the use of bioactive glass of varying particulate size, including a remineralizing amount of bioactive glass for remineralization of teeth, sealing fissures and/or pits, lining tooth structure, treating decay, capping pulp, treating sensitive post surgical tooth structure, sealing dentinal tubules and providing a surface for tissue regeneration. Litkowski et al. indicates that the use of bioactive glass particles in the size ranges disclosed produce a stable crystalline hydroxy carbonate apatite layer deposited onto and into the dentin tubules to obtain the desired effects.

Likewise, U.S. Pat. No. 5,735,942, issued to Litkowski et al. (1998), describes the use of varying sizes of bioactive glass particles for treatment of dentin hypersensitivity and occluding dentinal tubules. The bioactive glass compositions described form a rapid and continuous reaction with body fluids due to the immediate and long term ionic release of Ca and P from the core silica particles to produce a stable crystalline hydroxy carbonate apatite layer deposited onto and into the dentin tubules for the immediate and long term reduction of dentin hypersensitivity.

The bioactive glass as used in the Stoor patent is preferably administered as a composition comprising particles of bioactive glass admixed into water or an aqueous solution. Especially preferable is a paste comprising about 40 to 80 weight % of bioactive glass. The composition is to be administered and remain in the oral cavity for 10 minutes. The examples show that the composition of Stoor et al. results in an increase of pH of the bioactive glass mixture, when exposed to water, from 6.9 to about 10.8 after 10 minutes. Other tests showed a pH increase from 7 to 11 within 60 minutes upon use of bioactive glass particles less than 45 microns in size. Stoor et al. states that the antibacterial effect of the bioactive glass on the microorganisms tested may be due to high pH, osmotic effects and the Ca²⁺ concentration.

The utilization of a material which has a pH of about 10-11 which is applied for a prolonged period in the mouth is unworkable for use on a routine, daily basis. First, the high pH will irritate the oral tissues with prolonged exposure and will likely cause some tissue sloughing and pain to the patient. Second, patient compliance is likely to be minimal if one is required to maintain contact of the bioactive glass particles for any period of time greater than what is normally expected with daily tooth brushing (by way of example, about 30 seconds to two minutes).

Thus, there is still a need in the art for methods and compositions for the prevention and reduction of plaque build-up and gingivitis which are satisfactory for long term use and are conducive to patient compliance.

### BRIEF SUMMARY OF THE INVENTION

In one aspect of the invention, a method for preventing or reducing plaque or plaque build-up in an individual is provided comprising contacting all or a portion of the individual's oral cavity with a non-aqueous composition comprising a carboxyvinyl polymer, a humectant, a polyethylene glycol and about 0.25 to about 10% by weight bioactive glass particles having an average particle size of less than about 20 microns for a time effective to prevent or reduce plaque or plaque build-up. The non-aqueous composition may optionally contain a dentally acceptable abrasive. All or a portion of an individual's oral cavity is typically contacted with the non-aqueous composition for the amount of time generally used to brush one's teeth. Preferably, the contacting will be continued for more than about 30 seconds. In a preferred embodiment, the contacting will be continued for between about 30 seconds and about 2 minutes. Preferably, the contacting will take place daily, particularly one to three times per day.

In another aspect of the invention, a method for preventing or reducing gingivitis in an individual is provided comprising contacting all or a portion of the individual's oral cavity with a non-aqueous composition comprising a carboxyvinyl polymer, a humectant, a polyethylene glycol and about 0.25 to about 10% by weight bioactive glass particles having an average particle size of less than about 20 microns for a time effective to prevent or reduce gingivitis. The non-aqueous composition may optionally contain a dentally acceptable abrasive. All or a portion of an individual's oral cavity is typically contacted with the non-aqueous composition for the amount of time generally used to brush one's teeth. Preferably, the contacting will be continued for more than about 30 seconds. In a preferred embodiment, the contacting will be continued for between about 30 seconds and about 2 minutes. Preferably, the contacting will take place daily, particularly one to three times per day.

In a further aspect of the invention, a non-aqueous composition for preventing or reducing plaque or plaque build-up on teeth is provided comprising about 0.25 to about 10% by weight bioactive glass particles having an average particle size of less than about 20 microns in a non-aqueous carrier wherein the non-aqueous composition has a pH of about 6.0 to about 8.0, and wherein the pH of the non-aqueous composition increases less than about 1.5 pH unit upon exposure of the non-aqueous composition to an oral environment.

In a further aspect of the invention, a non-aqueous composition for preventing or reducing gingivitis is provided comprising about 0.25 to about 10% by weight bioactive glass particles having an average particle size of less than about 20 microns in a non-aqueous carrier wherein the non-aqueous composition has a pH of about 6.0 to about 8.0, and wherein the pH of the composition increases less than about 1.5 pH unit upon exposure of the composition to an oral environment.

### DETAILED DESCRIPTION OF THE INVENTION

The present invention provides non-aqueous compositions containing bioactive glass particles which compositions prevent or reduce plaque, plaque build-up and/or gingivitis. The non-aqueous compositions of the invention may be incorporated into oral hygiene compositions including a dentifrice such as a toothpaste or a composition to be applied by a dentist as a paste.

Surprisingly, it has been discovered that when low levels of small bioactive glass particles are included in amounts from about 0.25 to about 10% by weight in non-aqueous formulations, the resulting compositions are stable, pass ISO (International Organization for Standardization) standards, and may be used as effective dentifrice compositions. These compositions have unexpectedly high levels of antimicrobial activity against oral pathogens when placed in the oral cavity and, by way of example, clinical studies have demonstrated that these formulations are effective at reducing gingivitis and plaque when used in a standard daily routine of normal tooth brushing twice daily. Unexpectedly, the pH of the non-aqueous compositions of the invention does not increase to deleterious levels upon application of the bioactive glass containing non-aqueous compositions to the teeth and/or gums of an individual.

As used herein, the term “plaque” means sticky material that develops on and around the exposed portions of teeth, consisting of material such as bacteria, mucus and food debris. The term “plaque build-up” means plaque which remains on the teeth after one or more routine brushings of the teeth.

As used herein, the term “preventing plaque” means precluding the development of plaque on and around the exposed portions of teeth or reducing the risk of plaque forming on and around the exposed portions of teeth.

As used herein, the term “reducing plaque” means decreasing or lessening the amount of plaque forming on and around the exposed portions of teeth.

As used herein, the term “preventing plaque build-up” means precluding the development of plaque which remains on teeth after one or more routine brushings of the teeth or reducing the risk of plaque remaining on the teeth after one or more routine brushings of the teeth.

As used herein, the term “reducing plaque build-up” means decreasing or lessening the total amount of plaque remaining on one or more teeth after one or more routine brushings of the teeth.

As used herein, “gingivitis” means inflammation of the gums or gingiva due to bacteria-containing plaque on one or more adjacent teeth.

As used herein, “preventing gingivitis” means precluding the development of inflammation of the gums or gingiva due to bacteria-containing plaque on one or more adjacent teeth or reducing the risk of inflammation of the gums or gingiva due to bacteria-containing plaque on one or more adjacent teeth.

As used herein, “reducing gingivitis” means decreasing or lessening any inflammation of the gums or gingiva due to bacteria-containing plaque on one or more adjacent teeth.

As used herein, the term “non-aqueous” means anhydrous or substantially free of water. The individual components of the non-aqueous composition may contain limited amounts of water as long as the overall composition remains substantially free of water.

As used herein, the term “dentifrice” includes any preparation used in cleansing all or a portion of the oral cavity of an individual.

As used herein, the term “toothpaste” includes any semi-solid dentifrice preparation presented in the form of a paste, cream or gel specially prepared for the public for cleaning the accessible surfaces of teeth.

As used herein, the term “oral cavity” means an individual's teeth, and gums, including all periodontal regions including teeth down to the gingival margins and/or the periodontal pockets.

As used herein, the term “average particle size” in general means that some particles will be smaller and some particles will be bigger than the size specified. For purposes of this application and by way of example, where a non-aqueous composition contains bioactive glass particles of an average particle size of less than about 10 microns, typically 90-95% of the particles will be less than about 20 microns. Where the non-aqueous composition contains bioactive glass particles of an average particle size of less than about 5 microns, typically 90-95% of the particles will be less than about 15 microns. Where the non-aqueous composition contains bioactive glass particles of an average particle size of less than about 2 microns, typically 90-95% of the particles will be less than about 6 microns.

As used herein the terms “bioactive glass” or “biologically active glass” mean an inorganic glass material having an oxide of silicon as its major component and which is capable of bonding with growing tissue when reacted with physiological fluids. By way of example, a bioactive glass in accordance with the invention is a glass composition that will form a layer of hydroxycarbonate apatite in vitro when placed in a simulated body fluid. A bioactive glass as used herein is also biocompatible such that it does not trigger an overwhelmingly adverse immune response in the body, such as in the oral cavity.

Bioactive glasses are well known to those skilled in the art, and are disclosed, for example, in An Introduction to Bioceramics, L. Hench and J. Wilson, eds. World Scientific, New Jersey (1993), the entire contents of which is hereby incorporated by reference.

Typically, the compositions of the invention include particulate bioactive and biocompatible glass with a composition as follows: between about 40 and about 86% by weight of silicon dioxide (SiO₂), between about 0 and about 35% by weight of sodium oxide (Na₂O), between about 4 and about 46% by weight calcium oxide (CaO), and between about 1 and about 15% by weight phosphorus oxide (P₂O₅). Preferably, the glass includes between about 40 and about 60% by weight of silicon dioxide (SiO₂), between about 10 and about 30% by weight of sodium oxide (Na₂O), between about 10 and 30% by weight calcium oxide (CaO), and between about 2 and 8% by weight phosphorus oxide (P₂O₅). The oxides can be present as solid solutions or mixed oxides, or as mixtures of oxides.

CaF₂, B₂O₃, Al₂O₃, MgO and K₂O may be included in the composition in addition to silicon, sodium, phosphorus and calcium oxides. The preferred range for CaF₂ is between about 0 and about 25% by weight. The preferred range for B₂O₃ is between about 0 and about 10% by weight. The preferred range for Al₂O₃ is between about 0 and about 4% by weight. The preferred range for K₂O is between about 0 and about 8% by weight. The preferred range for MgO is between about 0 and about 5% by weight.

The most preferred glass is NovaMin®, also known as 45S5 Bioglass®, which has a composition including about 45% by weight silicon dioxide, about 24.5% by weight sodium oxide, about 6% by weight phosphorus oxide, and about 24.5% by weight calcium oxide.

Particulate, non-interlinked bioactive glass is preferred in the present invention. That is, the glass is in the form of small, discrete particles, rather than a fused matrix of particles or a mesh or fabric (woven or non-woven) of glass fibers. Note that under some conditions the discrete particles of the present invention may tend to cling together because of electrostatic or other forces but are still considered to be non-interlinked. The average particle size is typically less than about 20 microns, preferably less than about 15 microns, more preferably, less than about 10 microns, even more preferably less than about 5 microns, and ideally, about 2 microns. Particle size, as used herein, is measured by SEM or other optical microscopy techniques, or by laser light scattering techniques (i.e., using a Coulter counter).

The glass composition can be prepared in several ways, to provide melt-derived glass, sol-gel derived glass, and sintered glass particles. The sintered particles can be in sol-gel derived, or pre-reacted melt derived form. Sol-gel derived glass is generally prepared by synthesizing an inorganic network by mixing metal alkoxides in solution, followed by hydrolysis, gelation, and low temperature (around 200-900° C.) firing to produce a glass. Sol-gel derived glasses produced this way are known to have an initial high specific surface area compared with either melt-derived glass or porous melt-derived glass. Melt-derived glass is generally prepared by mixing grains of oxides or carbonates, melting and homogenizing the mixtures at high temperatures, typically between about 1250 and 1400° C. The molten glass can be fritted and milled to produce a small particulate material.

The bioactive glass particles are preferably melt-derived. In each preparation, it is preferred to use reagent grade glass and/or chemicals, especially since the glass and/or chemicals are used to prepare materials which ultimately are used in the oral cavity.

The amount of bioactive glass particles in the non-aqueous composition of the invention typically will be about 0.25 to about 10% by weight. Preferably, the amount of bioactive glass particles in the non-aqueous composition of the invention will be about 1 to about 10% by weight. In a more preferred embodiment, the amount of bioactive glass particles in the non-aqueous composition of the invention will be about 1 to about 7% by weight. In an even more preferred embodiment, about 2 to about 5% by weight bioactive glass particles are used in the non-aqueous composition.

The non-aqueous compositions of the invention include bioactive glass particulates in a non-aqueous carrier. Because of the reactivity of bioactive glass particulates when exposed to an aqueous environment, most common ingredients used for making dentifrices are not appropriate for containing the bioactive glass. For example, it has been discovered that, when using an aqueous based dentifrice and adding even 2.5% bioactive glass particles, within a few months, the pH of the dentifrice compositions will rise to over 11, which is unacceptable for various international standards for commercial toothpastes (BS 5136:1981 and ISO 11609:1995, for example, limit the pH to 10.5). However, in the non-aqueous compositions of the present invention, the pH does not increase to a deleterious level on the shelf or in the oral cavity. Without being bound by any theory, it is believed that through the use of low levels of small bioactive glass particles as described herein in a non-aqueous carrier, the desired antimicrobial effect with attendant prevention and/or reduction in plaque, plaque build-up and/or gingivitis can be obtained without a rise in pH detrimental to tissues of the oral cavity.

The non-aqueous compositions of the invention may include any suitable non-aqueous carrier which is substantially nonreactive with bioactive glass particulates and may be used in a dentifrice composition. Such non-aqueous carrier formulations are described, for example, in U.S. Pat. No. 5,882,630, issued to Gates et al. (1999), the contents of which are hereby incorporated herein in their entirety.

Non-aqueous compositions useful in the present invention preferably include a non-aqueous dentifrice carrier comprising a carboxyvinyl polymer, a humectant and a polyethylene glycol. Optionally, a dentally acceptable abrasive may be used in the non-aqueous dentifrice carrier. The non-aqueous composition additionally comprises bioactive glass particulates.

Suitable carboxyvinyl polymers for use in non-aqueous compositions of the invention are copolymers of acrylic acid cross-linked with polyallylsucrose, for example, carbomers such as Carbopol 974 and 934, or cross-linked with divinyl glycol, for example, Noveon AA-1. Carbopol™ polymers are manufactured by B.F. Goodrich Company. Carbopol™ 974 is preferred.

The carboxyvinyl polymer may be present in the range of from about 0.1 to about 7.5% w/w, preferably from about 0.3 to about 1.0%, more preferably about 0.35% by weight of the non-aqueous composition.

Suitable humectants for use in the present invention include glycerin, sorbitol, propylene glycol or mixtures thereof. It is well known that commercially available glycerin may contain between 0.5-2.0% by weight of water which is in association with the glycerin. Typically this amount is between 0.5-1.0% by weight. This small amount of water is bound to the glycerin and is therefore not available to the other ingredients. The skilled person would still consider a composition containing glycerin as being non-aqueous. The humectants should in any case be as anhydrous as possible and preferably used in solid form.

Glycerin is a preferred humectant.

As the humectant is used to make the formulations up to 100%, the humectant may be present in the range of from about 20 to about 90% by weight of the non-aqueous composition. Preferably the humectant is present from about 35 to about 75%, more preferably from about 45 to about 70% by weight of the non-aqueous composition.

The polyethylene glycol is selected so that it will substantially reduce any stickiness from the formulation and give a substantially smooth textured product. Suitably, the polyethylene glycol will be selected from PEG 300 and PEG 400. PEG 400 is preferred.

Advantageously, the polyethylene glycol is present in the range of from about 0.1 to about 40%, preferably about 15 to about 20% by weight of the non-aqueous composition.

Preferably, and in order to produce a product that is smooth and does not show any signs of stickiness, use of a particular ratio of carboxyvinyl polymer to polyethylene glycol is desirable.

Advantageously, the ratio of carboxyvinyl polymer to polyethylene glycol is in the range of about 1:15 to about 1:20, preferably about 1:17.5.

A dentally acceptable abrasive may optionally be added to the non-aqueous composition. Advantageously, the presence or absence of a dentally acceptable abrasive as well as the amount of such abrasive may be used to selectively control the abrasivity of the dentifrice composition made with the non-aqueous compositions of the invention. By way of example, the bioactive glass particles already present in the composition may provide an acceptable amount of abrasivity for the non-aqueous composition depending upon the ultimate use. By further way of example, a desired amount of dentally acceptable abrasive may be added to increase the abrasivity of the overall non-aqueous composition.

Suitable abrasives for use in the non-aqueous composition include, for example, amorphous, gelled, precipitated or fumed silica, zinc orthophosphate, sodium bicarbonate (baking soda), plastic particles, alumina, hydrated alumina, calcium carbonate, calcium pyrophosphate, insoluble metaphosphates or mixtures thereof.

The silica abrasive may be a natural amorphous silica, for instance diatomaceous earth; or a synthetic amorphous silica such as a precipitated silica. By way of example, the silica abrasive may be Syloid 63.

Generally, an amount of abrasive suitable for use in the non-aqueous composition of the present invention will be empirically determined to provide an acceptable level of cleaning and polishing, in accordance with the techniques well known in the art. Suitably, the abrasive will be present in from about 0 to about 60%, preferably from about 5 to about 30%, by weight of the non-aqueous composition.

Advantageously, a thickening agent is present in the formulation to give the product a rheology closer to that of a conventional dentifrice. Preferably, the thickening agent is an inorganic thickener. Suitably, the thickening agent is a thickening silica, for instance, Syloid 244FP.

The thickening silica will be in the range of from about 0.01 to about 10%, preferably about 2.0 to about 7.0% by weight of the non-aqueous composition.

The non-aqueous composition may additionally optionally contain other agents conventionally used in dentifrice formulations. Typically, these optional agents should not adversely affect the pH or reactivity of the overall non-aqueous composition. Such agents may include, by way of example, coloring agents, whitening agents such as titanium dioxide, flavoring agents, sweetening agents such as saccharin, cyclamate or acesulfame K, breath freshening agents such as sodium bicarbonate, foaming agents such as sodium lauryl sulfate, or preservatives.

In general, the optional agents may be used in a minor amount or proportion of the overall formulation. By way of example, such components are usually present in from about 0.001 to about 5% by weight of the non-aqueous composition.

In a preferred aspect of the invention, a dentifrice composition is prepared with the following components in percent by weight:

| | |
|---|---|
| Glycerin | about 50 to about 60 |
| Polyethylene glycol | about 15 to about 18 |
| Abrasive SiO₂ | about 10 to about 15 |
| Thickening SiO₂ | about 2 to about 5 |
| Titanium oxide | about 1 |
| Carbomer | about 0.2 to about 0.4 |
| Acesulfame K | about 0.4 |
| Bioactive glass | about 1 to about 10 |

The dentifrice composition typically will have a viscosity suitable for application to the oral cavity. The viscosity will vary depending on the type of dentifrice composition made and the ultimate use thereof. One of skill in the art can readily prepare compositions with suitable viscosities for use in the oral cavity from the teachings provided herein.

The initial pH of the non-aqueous compositions generally are about 6.0 to about 8.0. The pH of the composition after contact with saliva or other materials in the oral cavity typically will not rise more than about 1.5 pH unit. It has now been discovered that the relatively small increase in pH observed with the non-aqueous dentifrice compositions of the invention is advantageous due to the use of such compositions in the oral cavity on a daily basis and yet the compositions are effective to prevent and/or reduce plaque, plaque build-up and/or gingivitis.

Embodiments of the invention will be further explained by the following illustrative examples that are intended to be non-limiting.

### EXAMPLE 1

The objective of this study was to determine the antimicrobial properties of a number of bioactive glass (NovaMin®) containing dentifrice formulations against a number of common oral pathogens, and to compare these to a commercially available fluoride dentifrice as negative control.

The microbes used in the study were S. mutans (ATCC #25175), S. sanguinis (ATCC #10556), F. nucleatum (ATCC #10953) and A. neaslundii (ATCC # 19039). The bacteria were grown in DE broth to a concentration of at least 10⁶ CFU/ml. The bioactive glass (NovaMin®) was ground to an average particle size of 12 μm. Experimental dentifrices incorporating the bioactive glass particulate were formulated using a non-aqueous carrier based on glycerin. The compositions used are shown in Table II. The bioactive glass was added in either 3% w/w or 10% w/w. A commercial, fluoride containing dentifrice was used as a control (Colgate™ regular formula) The test articles were diluted 1:3 in distilled water, and the bacterial colonies were inoculated with the test articles and mixed gently for 30 seconds. At two minutes after inoculation, aliquots were taken and plated on Brain Heart Infusion Agar for three days for the aerobic bacteria and seven days for the anaerobic species. Viable CFUs were visually counted. All experiments presented represent the average of three replicates.

Table I shows the average log reduction in CFU's of the two minute exposure to the various test articles. The bioactive glass containing test groups showed significant levels of bacterial reduction compared to the control dentifrice.

TABLE I
Log Reduction in Bacterial CFU's

| | S. | S. | F. | A. |
|---|---|---|---|---|
| SAMPLE | Mutans | Sanguinis | nucleatum | naeslundii |
| 3% NM Dentrifice | 5.2 | 8.3 | 4.5 | 6.0 |
| 10% NM Dentrifice | 5.7 | 8.3 | 4.7 | 5.9 |
| Control Dentrifice | 1.4 | 3.0 | 1.3 | 1.1 |

TABLE II
Compositions

| | | | NovaMin ® |
|---|---|---|---|
| | 3% NovaMin ® | 10% NovaMin ® | Clinical Study |
| | Ex. 1 | Ex. 1 | Ex. 2 |
| Glycerin | 57.75% | 57.75 | 55.25 |
| PEG 400 | 17.50 | 17.50 | 18.00 |
| K Acesulfame | 0.40 | 0.40 | 0.40 |
| Carbopol | 0.40 | 0.40 | 0.40 |
| TiO₂ | 1.00 | 1.00 | 1.00 |
| Syloid 63 | 13.00 | 6.00 | 15.00 |
| Flavor | 0.85 | 0.85 | 0.85 |
| Na Lauryl S | 1.10 | 1.10 | 1.10 |
| Syloid 244FP | 5.00 | 5.00 | 3.00 |
| NovaMin ® | 3.00 | 10.00 | 5.00 |

### EXAMPLE 2

The objective of this experiment was to evaluate the anti-gingivitis and anti-plaque efficacy of a dentifrice containing a bioactive glass particulate and a negative control dentifrice without bioactive glass in a six week clinical trial.

The study design was a randomized, double-blinded, controlled clinical trial. The protocol was reviewed and approved by the Ethical committee of the Wuhan University, Wuhan Province, China. The Ethics Committee approval was by the Hubei Committee for Oral Health and the study was conducted in the School and Hospital of Stomatology, Wuhan University, China.

One hundred (100) volunteers took part in the study according to the inclusion and exclusion criteria. The subjects received a supragingival prophylaxis to remove all plaque, calculus and extrinsic stain. Following the baseline examination subjects were instructed to brush with their assigned dentifrice and toothbrush. The control dentifrice was the non-aqueous formulation of Table II without any bioactive glass particulate. Abrasive silica was added instead of the NovaMin® particles. The experimental dentifrice was formulated as a non-aqueous paste containing 5% by weight of bioactive glass particles with an average particle size of 12 μm. The composition used is detailed in Table II.

The levels, of Silness & Loe Plaque Index (PLI) and Gingival Bleeding Index (PBI) were determined at baseline (BL) and six weeks. A Student t-test was used to compare the effect between the test and control groups, p value was set at 5% level.

Ninety-five subjects (age range 20-48) finished the study. The PBI (BL, 1.14±0.79, 6 week 0.47±0.36) and PLI (BL 1.54±0.34, 6 week 1.29±0.40) were significantly reduced over the six week period in the test group (n=47) by 58.8% and 16.4% respectively while there was no difference of the PBI (BL 1.18±0.71, 6 week, 1.02±0.56) and PLI (BL 1.60±0.37, 6 week 1.57±0.41) for the control group (n=48).

This study demonstrated that a dentifrice containing bioactive glass in a non-aqueous formulation as detailed significantly improved oral health as measured by a reduction in gingival bleeding and reduction in supra-gingival plaque compared with bioactive glass free dentifrice over the six week study period.

### EXAMPLE 3

The objective of this study was to determine the antimicrobial properties of NovaMin® particulate used in dentifrice formulations, tested against one of the main pathogens associated with periodontal disease, A. actinomycetemcomitans (ATCC # 29523) at various concentrations of NovaMin® particulate.

The bacteria was grown in DE broth to a concentration of at least 10⁶ CFU/ml. The bioactive glass (NovaMin®) was ground to an average particle size of 2 μm. The bioactive glass was added at concentrations of 5%, 1.0%, 0.5%, and 0.1%. The test articles were diluted 1:3 in distilled water, and the bacterial colonies were inoculated with the test articles and mixed gently for 30 seconds. At various times after inoculation, (2, 5, and 60 minutes) aliquots were taken and plated on Brain Heart Infusion Agar for ten days. Viable CFU's were visually counted. All experiments presented represent the average of three replicates.

Table III shows the average log reduction in CFU's of the different time exposures to the NovaMin® particulate.

TABLE III
Log Reduction in Bacterial CFU's of A. actinomycetemcomitans

SAMPLE

| | 5.0% | 1.0% | 0.5% | 0.1% |
|---|---|---|---|---|
| 2 minute exposure | 6.4 | 0.7 | NR | NR |
| 5 minute exposure | >6.7 | 2.0 | 0.3 | NR |
| 60 minute exposure | >6.7 | 4.9 | 3.1 | NR |

The results of the study demonstrate significant and rapid reduction of the pathogen within 2 minutes at a dose of 5% NovaMin®, and a significant reduction in viability of 2 log for a 1% concentration of NovaMin® at a 5 minute exposure. There was also a significant reduction in the viability of the organism exposed to a 0.5% concentration of NovaMin® at a 60 minute exposure.

While the invention has been described in detail and with reference to specific embodiments thereof, it will be apparent to one skilled in the art that various changes and modifications can be made without departing from the spirit and scope of the invention

1. A method for preventing or reducing plaque or plaque build-up in an individual comprising contacting all or a portion of the individual's oral cavity with a non-aqueous composition comprising a carboxyvinyl polymer, a humectant, a polyethylene glycol and about 0.25 to about 10% by weight bioactive glass particles having an average particle size of less than about 20 microns for a time effective to prevent or reduce plaque or plaque build-up.

2. The method of claim 1 wherein the contacting is continued for more than about 30 seconds.

3. The method of claim 1 wherein the contacting is continued for about 30 seconds to about 2 minutes.

4. The method of claim 1 wherein the carboxyvinyl polymer is a carbomer.

5. The method of claim 1 wherein the humectant is glycerin, sorbitol, propylene glycol or mixtures thereof.

6. The method of claim 1 wherein the non-aqueous composition further comprises a dentally acceptable abrasive.

7. The method of claim 6 wherein the dentally acceptable abrasive is a silica abrasive.

8. The method of claim 1 wherein the non-aqueous composition further comprises a thickening silica.

9. The method of claim 1 wherein the non-aqueous composition comprises about 2 to about 5% by weight bioactive glass particles.

10. A method for preventing or reducing gingivitis in an individual comprising contacting the individual's oral cavity with a non-aqueous composition comprising a carboxyvinyl polymer, a humectant, a polyethylene glycol and about 0.25 to about 10% by weight bioactive glass particles having an average particle size of less than about 20 microns for a time effective to prevent or reduce gingivitis.

11. The method of claim 10 wherein the contacting is continued for more than about 30 seconds.

12. The method of claim 10 wherein the contacting is continued for about 30 seconds to about 2 minutes.

13. The method of claim 10 wherein the carboxyvinyl polymer is a carbomer.

14. The method of claim 10 wherein the humectant is glycerin, sorbitol, propylene glycol or mixtures thereof.

15. The method of claim 10 wherein the non-aqueous composition further comprises a dentally acceptable abrasive.

16. The method of claim 15 wherein the dentally acceptable abrasive is a silica abrasive.

17. The method of claim 10 wherein the non-aqueous composition further comprises a thickening silica.

18. The method of claim 10 wherein the non-aqueous composition comprises about 2 to about 5% by weight bioactive glass particles.

19. A non-aqueous composition for preventing or reducing plaque or plaque build-up on teeth comprising about 0.25 to about 10% by weight bioactive glass particles having an average particle size of less than about 20 microns in a non-aqueous carrier wherein the non-aqueous composition has a pH of about 6.0 to about 8.0, and wherein the pH of the non-aqueous composition increases less than about 1.5 pH unit upon exposure of the non-aqueous composition to an oral environment.

20. The non-aqueous composition of claim 19 wherein the non-aqueous carrier comprises carboxyvinyl polymer, a humectant, a polyethylene glycol, and a thickening agent.

21. The non-aqueous composition of claim 20 wherein the carboxyvinyl polymer is a carbomer.

22. The non-aqueous composition of claim 20 wherein the humectant is glycerin, sorbitol, propylene glycol or mixtures thereof.

23. The non-aqueous composition of claim 20 wherein the non-aqueous carrier further comprises a dentally acceptable abrasive.

24. The non-aqueous composition of claim 23 wherein the dentally acceptable abrasive is a silica abrasive.

25. The non-aqueous composition of claim 20 wherein the thickening agent is a thickening silica.

26. A non-aqueous composition for preventing or reducing gingivitis comprising about 0.25 to about 10% by weight bioactive glass particles having an average particle size of less than about 20 microns in a non-aqueous carrier wherein the non-aqueous composition has a pH of about 6.0 to about 8.0, and wherein the pH of the non-aqueous composition increases less than about 1.5 pH unit upon exposure of the composition to an oral environment.

27. The non-aqueous composition of claim 26 wherein the non-aqueous carrier comprises carboxyvinyl polymer, a humectant, a polyethylene glycol and a thickening agent.

28. The non-aqueous composition of claim 27 wherein the carboxyvinyl polymer is a carbomer.

29. The non-aqueous composition of claim 27 wherein the humectant is glycerin, sorbitol, propylene glycol or mixtures thereof.

30. The non-aqueous composition of claim 27 wherein the non-aqueous carrier further comprises a dentally acceptable abrasive.

31. The non-aqueous composition of claim 27 wherein the thickening agent is a thickening silica.

32. A non-aqueous dentifrice composition comprising about 50 to about 60% by weight glycerin, about 15 to about 18% by weight polyethylene glycol, about 10 to about 15% by weight abrasive silica, about 2 to about 5% by weight thickening silica, about one percent by weight coloring agent, about 0.2 to about 0.4% by weight carbomer, about 0.4% by weight sweetening agent and about 1 to about 10% by weight bioactive glass having an average particle size of less than about 15 microns, wherein the pH of the composition increases less than about 1.5 pH unit upon exposure of the composition to an oral environment.

33. The non-aqueous dentifrice composition of claim 32 comprising about 2 to about 5% by weight bioactive glass.

34. A toothpaste comprising glycerin, a polyethylene glycol, a thickening silica, a carbomer, and about 0.25 to about 10% by weight bioactive glass having an average particle size of less than about 20 microns, wherein the pH of the toothpaste increases less than about 1.5 pH unit upon exposure of the toothpaste to an oral environment.
